(19) United States
(12) Patent Application Publication
(10) Pub. No.: US 2002/0172710 A1
(43) Pub. Date: Nov. 21, 2002

(54) Clinical use of liposome technology for the delivery of nutrients to patients with the short bowel syndrome

(76) Inventor: Rebecca Wright Twine, Hempstead, NY (US)

Correspondence Address: Rebecca Wright Twine, 121 Cathedral Avenue, Hempstead, NY, 11550, US

(21) Appl. No.: 09/858,038

(22) Filed: May 16, 2001

(51) Int. Cl. A61K 9/127

(52) U.S. Cl. 424/450

(57) ABSTRACT

Short bowel syndrome may result from extensive resection of the small bowel. Because the transport of bile salts, cobalamin (vitamin B₁₂), and cholesterol is localized to the ileum, resection of this region is poorly tolerated. Patients who have had their ileum resected cannot absorb cholesterol, long chain fatty acids, fat soluble vitamins (A, D, E, and K), cobalamin (vitamin B₁₂), and the metal ions (Ca²⁺, Mg²⁺, and Zn²⁺). Liposomes are suitable biological vehicles for the non-parenteral delivery of these fat and water soluble nutrients directly into the blood of patients with the small bowel syndrome. Liposomes are compartmentalized vesicles consisting of bilayer lipids enclosing aqueous chambers. The lipid and water soluble nutrients can be commercially packaged in their respective compartments and delivered directly into the blood stream of these patients, thereby bypassing the intestines. The liposomes can pass through water barriers. Their solubility in lipid rich surface membranes permits the release of the enclosed nutrients and their diffusion into the interior of cells.

### CROSS REFERENCE TO RELATED APPLICATIONS

N/A

### STATEMENT REGARDING FEDERALLY SPONSORED RESEARCH OR DEVELOPMENT

N/A

### REFERNECE TO A MICROFICHE APPENDIX

N/A

### REFERENCE TO SEQUENCE LISTING, A TABLE, OR A COMPUTER PROGRAM LISTING APPENDIX

N/A

### BACKGROUND

1. Field of Invention

The invention relates generally to the short bowel syndrome, but it specifically relates to the delivery of nutrients via liposomes into the blood of patients who have had their ileum resected.

According to the literature, the small bowel has two primary functions, digestion and absorption of nutrients¹. The absorptive capacity of the small bowel is normally far in excess of need¹. However, the short bowel syndrome may develop after massive resection of the small intestine from trauma, mesenteric thrombosis, regional enteritis, radiation enteropathy, strangulated small bowel obstruction, neoplasm, necrotizing enterocolitis, and congenital atresia. The latter two are the most common pediatric causes¹. The short bowel syndrome is characterized by a group of signs and symptoms that result from a length of small bowel that is inadequate to support nutrition¹⁻³. The clinical hallmark of the disorder is diarrhea, fluid and electrolyte deficiency, and malnutrition¹⁻³.

The ability of a patient to maintain nutrition after extensive small bowel resection depends on the extent and the site of resection¹⁻³. Although many nutrients can be absorbed throughout the small intestine, but each nutrient has a major site of absorption³. When areas of the intestines are damaged or resected, the remaining intestines usually adapts effectively to absorb the nutrients that would normally have been absorbed by those areas¹⁻³. Because transport of bile salts, cobalamin (vitamin B₁₂), and cholesterol is localized to the ileum, resection of this region is poorly tolerated¹⁻³. The nutrients, cholesterol, vitamin B₁₂, and bile salts are exceptions to this adaptation mechanism. According to the literature, patients who have had their ileum resected can never actively absorb these three nutrients again¹⁻³.

Resection of specific segments of the small bowel leads to specific problems. Resection of the distal small bowel (the ileum) results in diarrhea, steatorrhea, and malabsorption¹⁻³. Conjugated bile salts are produced in the liver from cholesterol and are essential for normal fat absorption¹⁻⁴. It is reported in the literature that 90% of the bile salts are reabsorbed in the ileum and return to the liver via enterohepatic circulation⁴. In patients who have had their ileum resected, the bile salts are not reabsorbed¹⁻³. There is a profound interruption in the enterohepatic circulation of conjugated bile salts which results in a diminished pool and steatorrhea¹⁻³. Steatorrhea are fat-laden stools caused by the malabsorbption of fat¹⁻³.

The response to decreased reabsorption of bile salts is increased hepatic production of bile salts which leads to the diarrhea². The increased production of bile salts by the liver results in their excretion into the colon². The bile salts are irritants to the colon and cause a perpetuation of the diarrhea². Because the bile salts are not reabsorbed, the liver then must synthesize more bile salts, which lowers the intrahepatic free cholesterol pool⁴. As a result, hepatic LDL receptor synthesis is induced and more circulating low density lipid (LDL) is taken up by the liver⁴. The cholesterol component of the LDL is used to replenish the hepatic cell's pool of free cholesterol⁴. More than often, patients who have had their ileum resected have below normal blood levels of cholesterol because of increased synthesis of the bile salts.

The malabsorption syndrome refers to a clinical condition in which a number of nutrients and minerals are not normally absorbed¹⁻³. Almost always lipids fail to be absorbed, especially long chain fatty acids. Short and medium chain fatty acids (4-12 carbons) do not require bile salts for their absorption¹⁻⁴. They are absorbed directly into the intestinal epithelial cells. However, the absorption of long chain fatty acids require micelle formation by bile salts before they can be absorbed¹⁻⁴. The two essential long chain fatty acids, linoleate 18:2 (Cis Δ^{9,12}) and α linolenate 18:3 (Cis Δ^{9,12,15}) are not synthesized in the body⁵⁻⁶. They are required or “essential” in our diets because the body cannot synthesize fatty acids with these particular arrangements of double bond⁵⁻⁶.

Also, the fat soluble vitamins, A, D, K, and E require micellar solubilization by bile salts before they can be absorbed¹⁻³. These vitamins are not absorbed in patients who have had their ileum resected. In addition, the metal ions, Ca+², Mg+², and Zn+² are not adequately absorbed in these same individuals¹⁻³.

A major draw back in the current methodologies employed in the administration of those nutrients that cannot be absorbed by patients with the short bowel syndrome is the lack of a suitable non-parenteral vehicle for their delivery into the blood. Presently, the nutrients are delivered via hypodermic needles, indwelling nasointestinal tubes and/or venous catheters¹⁻³. The invasive procedures, particularly the insertion of large catheters into the jugular or subclavian veins of patients for the administration of nutrients are reserved mostly for hospitalized patients¹⁻². These procedures predispose patients to serious complications, for example, infections, phlebitis, air embolism, and metabolic abnormalities, etc¹. The invasive procedures are costly and are non-suitable for the long term delivery of nutrients to non-hospitalized patients and those who do not require lifetime parenteral nutritional support. Many non-hospitalized patients with the short bowel syndrome are deprived of the nutrients they cannot absorb, especially the fat soluble ones and metal ions. If these nutrients are not adequately replaced, patients with the short bowel syndrome will invariably and predictably develop the clinical signs of malnutrition and other serious health problems. Therefore, it is paramount that non-hospitalized patients and those who do not require lifetime parenteral nutritional support receive all of the nutrients they cannot absorb throughout their lives. Liposome technology is highly developed and has been perfected by many pharmaceutical companies. Liposomes have not been used clinically as non-parenteral vehicles for the delivery of those nutrients that cannot be absorbed by patients who have had their ileum resected. Liposomes are highly suitable vehicles for the non-parenteral delivery of fat and water soluble nutrients, and ions directly into the blood stream of these patients.

2. Prior Art

After hospitalized patients have undergone bowel resection for any cause, they may receive either of the following types of nutritional support: (1) enteral nutritional therapy (ENT) (2) parenteral nutritional therapy (PNT) and (3) peripheral nutritional therapy (PPN). Enteral diets are administered via a nasointestinal feeding tube with the tip positioned in the proximal duodenum of the small bowel¹. Nasointestinal feeding is used for short term, less than six weeks of nutritional therapy. The nutrients are administered continuously with a volumetric pump. Enteral feedings may be used as a supplement or the sole source of nutrients. There are two kinds of commercial preparations, standard and special , based upon protein content or clinical application¹. The standard diets are recommended for patients who have normal gut function, but are under considerable metabolic stress¹. The special enteral diets contain protein in the form of low-molecular weight free amino acids or polypeptides. Amino acids (elemental) and polypeptide diets are efficiently absorbed in the presence of compromised gut function¹. The most common types of enteral diets are either elemental diets (vivonex, Flexical) or polymeric diets (Isocal, Ensure)². The major disadvantage of the enteral diets are problems with increased osmolality. In addition, the special enteral diets may contain nutrients that cannot be absorbed by patients whose ileum had been resected.

Parenteral nutritional therapy (PNT) is administered to patients when their gastrointestinal tract cannot be used¹. Parenteral nutrition is more expensive and is associated with more technical , metabolic, and septic complications¹. This therapy is indicated in patients who require short term, less than 10 days of nutrition because they are unable to ingest adequate nutrition¹. It is contraindicated in patients who can be nourished via the gut. Parenteral nutrition bypasses the gut and is delivered directly into the blood stream. Peripheral parenteral nutrition is usually infused via an 18-gauge intravenous cannula in a peripheral vein¹. A 16-gauge single-lumen central venous catheter (SLCVC) positioned in the superior vena cava via the subclavian or the internal juglar vein is also used¹.

Total peripheral nutrition (TPN) is indicated for patients who cannot be nourished enterally and need more than 10 days of nutritional support¹. In patients over age ten, TPN solutions are routinely administered via a single-lumen central venous catheter (SLCVC) with a Luer-lok connection¹. The TPN catheter is inserted either into the superior vena cava via the internal juglar or subclavian vein or into the inferior vena cava via the femoral vein¹.

All three forms of the invasive nutritional support described above predispose already ill patients to serious technical, infectious, and metabolic complications. The complications of enteral tube feedings are technical, functional, and nutritional¹. The technical complications are tracheal, esophageal, bronchial, or duodenal perforations Functional complications are nausea and vomiting, etc¹. Major nutritional complications, especially in patients who have had their ileum resected are the malabsorption of cholesterol, long chain fatty acids, fat soluble vitamins, vitamin B₁₂, and the metal ions, Ca²⁺, Mg²⁺, and Zn²⁺. Peripheral parenteral nutrition predisposes patients to infection, for example, phlebitis, and septicemia. Some of the common technical complications of total parenteral nutritional therapy (TPN) are air embolism, pneumothorax , catheter site infections¹. The metabolic complications of TPN are hyperglycemia, acidosis, and electrolyte and mineral abnormalities¹.

Patients who have had their ileum resected and no longer require parenteral nutrition are prescribed oral high-carbohydrate, high protein, and low fat diets². The low fat component of the diet consists chiefly of medium chain fatty triglycerides (6-12 carbons)². Short and medium chain triglycerides can be absorbed in patients with the small bowel syndrome²⁻³. The short and medium chain triglycerides do not require bile salts for absorption²⁻³. They can pass directly into the venous portal system, transported as fatty acids bound to albumin. However, the long chain triglycerides (16-18 carbons) cannot be absorbed by patients with the small bowel syndrome. The major disadvantage of the low fat diet is that cholesterol, long chain fatty acids, including the essential fatty acids (linoleic and linolenate), fat soluble vitamins (A D,E, and K) cannot be absorbed in patients who have had their ileum resected¹⁻³. These nutrients are administered parenterally via indwelling venous catheters to patients with the short bowel syndrome¹. The current nutritional art does not provide a non-parenteral route for the delivery of the nutrients that cannot be absorbed in patients with the short bowel syndrome. Patients who have had their ileum resected cannot absorb cholesterol, long chain fatty acids, fat soluble vitamins (A, D, E, and K), vitamin B₁₂, and the metal ions (Ca²⁺, Mg²⁺, and Zn²⁺) via enteral or oral diets¹⁻³.

Patients with the short bowel syndrome require periodic intramuscular injections of Vitamin B₁₂ because they cannot absorb this water soluble vitamin²⁻³. There are no current maintenance nutritional therapies that provide for the adequate delivery of vitamin B₁₂ to patients with the short bowel syndrome other than by intravenous delivery or intramuscular injections. There are some vitamin B₁₂ oral and nasal sprays on the market . The manufacturers of these sprays claim that the B₁₂ is absorbed directly into the blood stream and bypasses the intestines if administered properly. There are also some similar oral sprays that contain trace metals, for example, zinc in which the manufacturers make the same claim. A major disadvantage of these sprays is that they usually will have only one nutrient or ion in them. The patients would have to purchase several different sprays containing each of the nutrients, for example, one for zinc, and one for vitamin B12. This could be costly. Also, such sprays would require different and multiple dosings by patients. In addition, these sprays don't allow for the absorption of fat soluble nutrients. The fat soluble nutrients have to be packaged in a form that permit their absorption directly into the blood stream. Individuals with the small bowel syndrome require the delivery of those nutrients they cannot absorb into their blood via a safe, reliable, predictable, and a non-invasive vehicle. Ambulatory patients who have had their ileum resected and are fairing well in life should not have to be hospitalized periodically to receive the nutrients they cannot absorb via nasointestinal tubes or intravenous catheters. In addition, these patients should not have to make frequent visits to the offices of medical doctors in order to receive intramuscular injections of some the nutrients they cannot absorb. It is noteworthy that there are not any available commercial preparations of vitamins E and D for intramuscular injection.

There is no prior art known to the inventor that utilizes liposome technology in the delivery of those nutrients that cannot be absorbed by patients who have had their ileum resected. Non-hospitalized patients who have had their ileum resected should not be deprived of any of the nutrients they cannot absorb because of the lack of a non-parenteral vehicle that would assure their delivery directly into the blood.

### BRIEF SUMMARY OF THE INVENTION

The primary objective of the invention is to deliver the nutrients, cholesterol, essential long chain fatty acids (linoleic and linolenate), fat soluble vitamins (A, D, E, and K), cobalamin (vitamin B₁₂), and the metal ions, Ca²⁺, Mg²⁺, and Zn²⁺ via liposomes directly into to the blood of patients with the short bowel syndrome.

A major advantage of utilizing liposomes as vehicles for the delivery of nutrients to patients with the short bowel syndrome is that they do not require the action of bile salts for their entry into cells; they can pass through water barriers; their solubility in lipid rich surface membranes of cells permits the release of the enclosed nutrients and their diffusion into the cells.

Another advantage of utilizing liposomes as vehicles for the delivery of nutrients to patients with the short bowel syndrome is that they accumulate in the liver, especially if they are unmodified (for example, not tagged with antibody); The liver is the primary site for the biosynthesis of fatty acids, cholesterol, bile salts, and serve as a storage site for vitamin A and B₁₂, etc.; cholesterol can be delivered directly to the liver via liposomes to replenish the diminished intrahepatic cholesterol pool caused by the increased synthesis of the bile salts in patients with the short bowel syndrome.

Still another advantage of utilizing liposome technology is that fat and water soluble nutrients can be delivered simultaneously into the blood stream of patients with the short bowel syndrome, eliminating multiple and different nutrient dosings.

A further advantage of utilizing liposomes in the delivery of nutrients to patients with the short bowel syndrome is the elimination of needles and indwelling intravenous catheters.

A still further advantage of utilizing liposomes in the delivery of nutrients to patients with the short bowel syndrome is that they are unlikely to produce allergic reactions; the carrier lipid is usually phosphatidylcholine (lecithin); phosphatidylcholine is a membrane lipid in human cells; phosphotidylcholine is plays important roles in biochemical reactions that occur in human cell membranes; phospatidylcholine is a natural substance.

Liposomes provide a safe, effective, and efficient method of delivering fat and water soluble nutrients to patients with the small bowel syndrome.

Another advantage of utilizing liposomes for the delivery of nutrients to patients with the small bowel syndrome is that they do not have to be administered by medical personnel, for example, the patient can administer the nutrients to themselves via an oral preparation.

A more further advantage of utilizing liposomes for the delivery of nutrients that cannot be absorbed by patients with the small bowel syndrome would be better patient compliance; patients with the small bowel syndrome would be more apt to engage in nutrient replacement therapy because they will not have the fear of receiving injections or the insertion of catheters into their veins; the nutrients can be taken in their own privacy.

Another advantage of utilizing liposomes for the delivery of nutrients into the blood of patients with the short bowel syndrome is that the lipid matrix of the structures are synthetic versions of naturally occurring lipids in the body and are therefore biocompatible and biogradable by the usual in vivo lipid metabolic pathways.

Still another advantage of the delivery of the fat and water soluble nutrients via liposomes into the blood of patients with the short bowel syndrome is that they would not be predisposed to the infectious and technical complications that can arise from parenteral nutritional therapy, for example, the insertion of intravenous catheters into veins of patients.

The delivery of nutrients via liposomes can supplement parenteral and/or enteral nutritional therapy.

### DETAILED DESCRIPTION OF THE INVENTION

The biochemical structure of liposomes make them excellent non-parenteral vehicles for the delivery of those fat and water soluble nutrients that cannot be absorbed in the intestines of patients who have had their ileum resected. In addition, the physical properties of the nutrients that cannot be absorbed allow them to be commercially incorporated into liposomes.

Lipids are small-water-insoluble biomolecules generally containing fatty acids, sterols, or isoprenoid compounds. Lipids are soluble in nonpolar solvents. Lipids cannot enter into the blood stream unpackaged because they would coalesce, impeding the flow of blood. Lipids must be packaged in a form that is soluble in blood. The commercial incorporation of the fat soluble nutrients, cholesterol, long chain fatty acids, and the fat soluble vitamins (A, D, E, and K) would simulate the physiological packaging of these same nutrients into chylomicrons by intestinal epithelial cells for their transport into the blood to tissues⁴⁻⁵. Liposomes and chylomicrons are similar in that they are soluble in the blood and can transport lipids to tissues⁵⁻⁷. Liposomes differ from chylomicrons in that they consist of a bilayer lipid membrane. Chylomicrons consist of a monolayer membrane of phospholipids. Liposomes also differ from chylomicrons in that they can transport both fat and water soluble nutrients.

Cholesterol, long chain fatty acids, and fat soluble vitamins require micellar formation before they can enter into the intestinal epithelial cells. A certain concentration of bile salts are required for micellar formation of these lipids⁴. Patients who have had their ileum resected cannot absorb long chain fatty acids, cholesterol, and fat soluble vitamins because they lack bile salts¹⁻³. Their bile salts are not reabsorbed, but excreted. Consequently, the dietary intake of these nutrients are not absorbed in the small bowel, but are excreted. Because of the physical characteristics of these fat soluble nutrients, they can be commercially packaged in liposomes and delivered directly into the blood of patients with the short bowel syndrome.

Liposome technology has been perfected⁵⁻⁷. Liposomes have numerous uses as biochemical and biophysical tools. Liposomes are used as vehicles for the delivery of both water and oil soluble materials into cells⁵⁻⁷. Liposome technology is based on the amphipathic characteristics of certain classes of lipids, especially the phosholipids⁵⁻⁸. Phospholipids form bilayer lipids that are similar to the membranes of human cells. Amphiphatic lipids contain both polar and nonpolar regions⁵⁻⁸. Phospholipids are composed of a hydrophobic (nonpolar) “tail” region, usually derived from long chain fatty acids, and a hydrophillic (polar) “head” region which contains a phosphate group with a negative charge, and in some instances a group with a positive charge⁵⁻⁸. The difference in the regions at the two ends of phospholipid molecules causes them to concentrate at interfaces between aqueous and non-aqueous phases within the cell. Their hydrophobic regions react with the lipids and their hydrophillic regions react with water. Membranes naturally tend to form closed structures, to avoid exposing the hydrophobic ends of lipid bilayers to the solvent. Liposomes can be made with membrane fragments or synthetic phospholipids⁵⁻⁷. They can be made to contain compounds buried in the membrane or totally enclosed⁵⁻⁷. Membrane bound components can be used to target the liposomes to the appropriate cells and fusion with the cell membranes delivers the contents of the liposome to the interior of cells.

When amphipathic lipids are mixed with water they form microscopic lipid aggregates in a phase separate from their aqueous surroundings⁵⁻⁷. The lipid molecules cluster together with their hydrophobic moieties in contact with each other and their hydrophillic groups interacting with the water. The hydrophobic interactions among lipid molecules provide the thermodynamic driving force for the formation and maintenance of these structures⁵⁻⁶. There are three types of lipid aggregates formed when amphipathic lipids are mixed with water (1) spherical micelles (2) a bilayer and (3) a liposome. In the micelles, the hydrophobic chains of the fatty acids are sequestered in the core of the sphere⁵⁻⁶. There is virtually no water in the interior of a micelle⁶. In a bilayer, all acyl side chains except those at the edges of the sheet are protected from the interaction with water⁵⁻⁶. When an extensive two-dimensional bilayer folds on itself, it forms a liposome, a three dimensional hollow vesicle enclosing an aqueous cavity.⁵⁻⁷ By forming vesicles, bilayer sheets lose their hydrophobic edge regions, thereby achieving maximal stability in their aqueous environment These bilayer vesicles enclose water, creating a separate aqueous compartment. Liposomes are also known as lipid vesicles. The membranes enclose a portion of the aqueous phase much like the cell membrane which encloses the cell⁵⁻⁸.

A widely used synthetic phospholipid for the commercial production of liposomes is phosphatidylcholine (lecithin)⁵⁻⁷. Phosphatidylcholine is an amphipathic phosphoacylglycerol⁴⁻⁸. It consists of an elongated nonpolar (hydrophobic “tail”), the two long fatty acyl chains and a polar (hydrophillic “head”) group. The two fatty acid molecules are esterfied to two hydroxy groups of glycerol, and a second alcohol, the head group, esterfied to the third hydroxyl of glycerol via a phosphodiester bond. When phospholipids are dispersed in water, they spontaneously form bilayer membranes. These bilayer membranes are also called lamellae. They are composed of two monolayer sheets of lipid molecules with their nonpolar (hydrophobic) surfaces facing each other and their polar (hydrophillic) surfaces facing the aqueous medium. Thus, the basic foundation of the liposome is a lipid bilayer⁵⁻⁷. In such a layer, the nonpolar hydrophobic tails of the phospholipid molecules point inward, forming a nonpolar zone in the interior of the bilayer⁵⁻⁷. The nonpolar zone surrounds the innermost aqueous (hydrophillic) compartment of the liposome⁵⁻⁷.

The simplest laboratory procedure for the formation of liposomes is the hand shaking of an aqueous solution of anhydrous lipids with water for seconds to hours, depending on the natures of the lipids or aqueous phases⁵⁻⁷. This simple procedure spontaneously yields large, multi-lamellar liposomes with diameter 1 to 10 micrometers, which are composed of a few to hundreds of concentric lipid bilayers alternating with layers of aqueous phases⁵⁻⁷.

Smaller unilamellar vesicles of 20 nanometer in diameter can be produced by subjecting multilamellar vesicles to high-frequency ultrasound waves^{6,7}. The sonicated mixture results in a dispersion of closed vesicles that are uniform in size. Vesicles can also be prepared by rapidly mixing a solution of lipid in ethanol with water⁶. This is usually done by injecting the lipid through a fine needle into an aqueous solution⁶. Vesicles formed by these methods are nearly spherical in shape and have a diameter about 500 angstrom units. Larger vesicle of 1 μm, in diameter can be prepared by slowly evaporating the organic solvent from a suspension of phospholipid in a mixed solvent system⁵⁻⁷. Fusion of small unilamellar vesicles by methods requiring particular lipids or stringent dehydration-hydration conditions can yield unilamellar vesicles as large or larger than cells⁵⁻⁷.

Ions or molecules can be trapped in the aqueous compartments of lipid vesicles (liposomes) by forming them in the presence of those water soluble substances desired to be delivered into other cells⁵⁻⁷. The vesicles can be loaded almost with any water-soluble molecule ⁵⁻⁷.

Liposome technology has advanced above and beyond the production of the conventional liposome^{9,10,11}. The development of multivesicular lipid-based carrier technology provides for sustained and controlled delivery of therapeutic agents^{9,10,11}. Also, the multivesicular technology permits high drug loading and the modulation of drug release rates¹². The biocompatibility of the liposome matrix allows for its delivery into sensitive areas of the body without a “foreign body response.^{9,10,11}” Current multivesicular liposome formulations have been shown to exhibit excellent storage stability^{9,10,11,12}. The multivesicular liposomes have been shown to be cost effective because they can be readily manufactured on a commercial scale¹³.

Liposomes appear to be the ideal biological vehicles for the delivery of fat and water soluble nutrients to patients with the short bowel syndrome who do not require parenteral nutrition. Most of the nutrients that are malabsorbed in these patients are processed and /or stored in the liver. It was previously mentioned that a major advantage of using liposomes for the delivery of those nutrients that cannot be absorbed by patients with the short bowel syndrome is that they accumulate in the liver, where they are usually stored and metabolized. It was also mentioned earlier that fat and water soluble nutrients can be commercially packaged together in liposomes and delivered simultaneously into the blood stream of patients with the small bowel syndrome.

The Rationale for the Delivery of Cholesterol via Liposomes to Patients with the Short Bowel Syndrome: Although cholesterol is an essential molecule in humans, it is not required in the diet⁴. It can be synthesized in the liver from the simple precursor, acetyl Coenzyme A⁴. However, patients with short bowel syndrome tend to have below normal cholesterol levels for several reasons. A major reason why patients with the short bowel syndrome have low blood cholesterol levels is due to its malabsorption. Bile salts are absorbed from the ileum together with dietary cholesterol⁴. They enter the portal circulation, which carries them to the liver, where they can be re-excreted into the duodenum (upper portion of the small bowel)⁴. Cholesterol requires micellar solubilization by the bile salts for absorption⁴. Bile salts are not reabsorbed in individuals who have had their ileum resected¹⁻³. Therefore, they cannot absorb dietary cholesterol⁴. The dietary cholesterol is lost in the feces.

Another reason why patients with the short bowel syndrome have low blood cholesterol levels is because of the increased demand on the intra-hepatic cholesterol pool for the biosynthesis of bile salts⁴. Bile salts are synthesized in the liver from cholesterol. According to the literature, a critical concentration of bile salts for micelle formation (5 to 10 μmmol per milliliter) is maintained by a very efficient enterohepatic circulation of bile salts⁴. It was stated in the literature that the total bile salt pool is about 2 to 4 grams and that about 95% of it is actively reabsorbed in the ileum and returned to the liver by the venous portal system⁴. It was further stated in the literature that approximately, 20 to 30 grams of bile salts recirculate in the enterohepatic circulation³. It was also reported in the literature that only about 200 to 600 milligrams of bile salts are excreted in the feces per day³. According to the literature, the amount of bile salts that is excreted into the feces daily must be replaced by the hepatic biosynthesis of cholesterol^{3,4}. The amount of bile salts excreted is much larger in patients with the short bowel syndrome. Since the bile salts have to be replaced by hepatic cholesterol, much of the cholesterol synthesized in the liver is utilized in the biosynthesis of bile salts. Therefore, increased synthesis of bile salts by the liver results in low blood levels of cholesterol in patients who have had their ileum resected.

The diarrhea that results from intestinal resection may be on the basis of the region removed, for example, the ileum with its special transport sites for active bile salt absorption or the length of the bowel resected. At least 50% of the small bowel is required in order to avoid diarrhea and malnutrition associated with the small bowel syndromes. The diarrhea in patients with the short bowel syndrome results from the excretory effects of malabsorbed bile salts on the colonic mucosa¹⁻³. The bile salts are irritants to the colonic mucosa²⁻³. Cholestyramine is the treatment of choice for the diarrhea in patients with the short bowel syndrome. Cholestyramine also causes a reduction in the serum cholesterol⁴. Cholestyramine binds bile salts and prevents their reabsorption⁴. The binding of the bile salts by cholestyramine leads to the increased conversion of cholesterol to bile salts⁴. This results in a further diminished cholesterol intrahepatic pool. In addition, bile salts are necessary for the absorption of cholesterol from the small bowel; hence there is also decreased absorption of cholesterol.

Cholesterol is a membrane constituent⁴. Cholesterol adds stability to the phospholipid bilayer of membranes⁴. All growing animal tissues need cholesterol for membrane synthesis, and some organs (adrenal gland and gonads), for example, use cholesterol as a precursor for steroid hormone production⁴⁻⁶. Cholesterol is a precursor of vitamin D⁴⁻⁶.

All steroid hormones are derived from cholesterol⁴⁻⁶. Two classes of steroid hormones are synthesized in the cortex of the adrenal gland: mineralcorticoids which control the reabsorption of inorganic ions (Na⁺, Cl⁻, and HC0₃−) by the kidney, and glucorticoids, which regulate gluconeogenesis and also reduce the inflammatory response. As previously mentioned, cholesterol serves as the precursor of bile salts, detergent-like compounds that function in the process of lipid digestion and absorption⁴.

Cholesterol is the precursor of all sex hormones⁴⁻⁶. The sex hormones are produced in the male and female gonads and the placenta. They include androgens (e.g., testosterone and estrogens (e,g., estradiol) which influence the development of secondary sexual characteristics in males and females, respectively, and progesterone, which regulates the reproductive cycle in females.

The low blood cholesterol levels in individuals with the small bowel syndrome must be replenished periodically with exogenous cholesterol, especially in those who are being treated with cholestyramine for the diarrhea. The additive effect of cholestyramine on an already diminished intrahepatic cholesterol pool can profoundly affect the health and well being of patients with the short bowel syndrome. Therefore, it is absolutely necessary that patients with the small bowel syndrome receive exogenous cholesterol replacement therapy, if required. Extemely low blood levels of cholesterol could adversely affect growth and reproductive maturity in pediatric patients with the small bowel syndrome.

Cholesterol is amphipathic⁵⁻⁶. It has a hydrophobic and a hydrophillic unit. It has a polar head group, the hydroxyl group at C-3 and a nonpolar hydrocarbon body (the steroid nucleus and the hydrocarbon side-chain at C-17)⁶. Cholesterol in its extended form is about as long as 16-carbon fatty acid⁶. According to the literature, free cholesterol inserts into bilayers with its long axis perpendicular to the plane of the membrane⁶. The hydroxyl group of cholesterol hydrogen bonds to a carbonyl oxygen atom of a phospholipid head group , whereas the hydrocarbon tail of cholesterol is located in the nonpolar core of the bilayer⁶. In cholesterol esters, the hydroxyl group is esterfied to a fatty acid. Chylomicrons transport both free cholesterol and cholesterol esters⁴. It was stated in the literature that the esterfication of cholesterol causes the molecule to become more hydrophobic. It was further stated in the literature that cholesterol esters are more readily packaged in lipoprotein particles, or lipid droplets in the cytosol of the cell. According to the literature, the bulk of cholesterol is transported in the form of cholesterol esters in chylomicrons and lipoproteins. The incorporation of cholesterol as esters into liposomes would simulate the physiological packaging and transport of cholesterol by chylomicrons. If cholesterol esters are commercially incorporated into liposomes, they would be sequestered into the hydrophobic interior of the structures.

Rationale for the Delivery of the Essential Long Chain Fatty Acids via Liposomes into the Blood of Patients with the Short Bowel Syndrome: Fatty acids are derived from the diet or synthesized mainly in the liver from glucose. The fatty acids are divided into four groups: short chains with 2 or 3 carbons, medium chains with 4-12 carbons, long chains with 21-20 carbons, and very long chains with more than 20 carbons. Long chain fatty acids with 14-20 carbons predominate in the body. It was previously mentioned that long chain fatty acids cannot be absorbed by patients who have had their ileum resected because they require micellar solubilization. These patients lack the necessary bile salts for micellar solubilization of long chain fatty acids. Consequently, they are not absorbed from their diets. They are excreted and contribute significantly to the steatorrhea characteristic of the short bowel syndrome.

The body cannot synthesize the two essential long chain fatty acids, linoleate and linolenate⁵⁻⁶. Because these two long chain fatty acids are necessary precursors for the synthesis of other products , they are essential fatty acids for humans. They must be obtained from plant material in the diet. Once ingested linoleate may be converted into certain other polyunsaturated fatty acids, particularly γ-linolenate, eicosatetraenoate (arachidonate) which can only be made from linoleate⁴⁻⁶. Arachidonate, 20: 4 (Δ^{5,8,11,14}), is an essential precursor of regulatory lipids, the eicosanoids. The prostaglandins, thromboxanes, and leukotrienes belong to this group of compounds. The eicosanoids are a family of very potent biological signaling molecules that act as short-range messengers affecting tissues near the cells that produce them⁴⁻⁵. This family of compounds are known to be involved in reproductive functions; in the inflammation, fever, and pain associated with injury or disease; in the formation of blood clots and the regulation of blood pressure; in gastric acid secretion; and in a variety of other processes important in human health and disease. Therefore, it is absolutely necessary that individuals who have had their ileum resected receive these two essential fatty acids.

Fatty acids are carboxylic acids with hydrocarbon chains of 4 to 36 carbons⁵. In some fatty acids, the chain is fully saturated (contains no double bonds). The physical properties of fatty acids, and of compounds that contain them, are largely determined by the length and degree of unsaturation of the hydrocarbon chain⁵ . The nonpolar hydrocarbon chain accounts for the poor solubility of fatty acids in water. The longer the fatty acyl chain and the fewer the double bonds, the lower the solubility in water⁵. The carboxylic acid group is polar (and ionized at neutral pH), and account for the slight solubility of short-chain fatty acids in water⁵. In vertebrate animals, free fatty acids (having a free carboxylate group) circulate in the blood bound to a protein carrier, serum albumin⁵. However, fatty acids are present mostly as carboxylic acid derivatives such as esters or amides⁵. In general, fatty acids that lack the charged carboxylate group are less soluble in water than are the free charged carboxylic acids⁵. The long chain fatty acids are essentially hydrophobic and require solubilization by bile salts before they can be absorbed⁵. The two long chain fatty acids, linoleic and linolenate can be incorporated into liposomes as ionized or esterified molecules. In either form, they would most likely occupy the hydrophobic interior of the liposome because of their poor solubility in water. Even if they were incorporated into liposomes in the ionized form, the double bonds associated with both essential fatty acids would not be enough to offset the strong hydrophobicity of the long hydrocarbon chains, (“tails” of 18 carbon atoms).

Perhaps, the possibility exists that the essential long chain fatty acids could be esterfied with cholesterol under laboratory conditions and incorporated into liposomes. Cholesterol contains 27 carbon atoms. It has 8 carbon atoms in its branched aliphatic side chain, and its steroid nucleus contains a double bond between carbons 5 and 6 and a hydroxyl group at position 3. The 3-OH group of cholesterol allows it to react with fatty acids. This hydroxy group can be esterfied to fatty acids, producing cholesterol esters. An ester linkage is formed when a carboxylic acid and an alcohol react, splitting out water⁴. In human liver cells, cholesterol esters are formed through the action of acyl-:cholesterol acyltransferase (ACAT). This enzyme is also located in cells, particularly, those needed to store cholesterol for the synthesis of steroid hormones⁴. The synthesis of cholesterol esters converts cholesterol into an even more hydrophobic form for storage and transport. Cholesterol esters occupy the hydrophobic interior of chylonicrons. If the essential long chain fatty acids are esterfied with cholesterol and incorporated into liposomes, they also would be sequestered into the hydrophobic interior of the structure. Cholesterol esters are transported to other tissues that use cholesterol, for example, the gonads for the synthesis of steroid hormones, or are stored in the liver⁴. The cholesterol esters can be hydrolyzed by liver endosomal enzymes into free cholesterol and fatty acids⁴.

The long chain fatty acids require the action of bile salts before they are packaged as triacyglycerols and incorporated into chylomicrons by intestinal epithelial cells. Triacyglycerols are fatty acid esters of glycerol. Triacylglycerols contain three fatty acid molecules esterfied to the three hydroxyl groups of glycerol. The triacylglycerols are primarily storage fats. The insertion of the long chain fatty acids as triacylglycerols into liposomes would simulate the normal intestinal packaging of them into chylomicrons. Because of the polar hydroxyls of glycerol and the polar carboxylates of the fatty acids are bound in ester linkages, triacylglycerols are nonpolar, hydrophobic molecules, essentially insoluble in water⁵. If the long chain fatty acids (linoleate and linolenate) are incorporated as triacylglycerols into liposomes, they would also be sequestered into the hydrophobic interior of the structures. Thus, their incorporation into liposomes as triacylglycerols simulate the packaging of those into chylomicrons by the intestinal epithelial cells.

Large amounts of triacylglycerols are stored in human cells called adipocytes, or fat cells. Many patients with the small bowel syndrome are underweight. The incorporation of long chain fatty acids as triacylglycerols into the liposomes could enable them to achieve an ideal body weight commensurate with their height and body frame.

The fat soluble vitamins A, D, E, and K are isoprenoid compounds⁵. Vitamin A is a pigment essential to vision. A deficiency in vitamin A may lead to night blindness. Vitamin D is a derivative of cholesterol and the precursor hormone essential in calcium and phosphate metabolism in vertebrate animals. A deficiency in vitamin D leads to defective bone formation resulting in rickets. Vitamin K is a lipid cofactor required for normal blood clotting. Vitamin K deficiency can result in defective coagulation. Vitamin E is the collective name for a group of closely related lipids called tocopherols, all of which contain a substituted aromatic ring and a long carbon side chain⁵. Vitamin E deficiencies can lead to a scaly skin disorder, muscular weakness and wasting, and sterility. Vitamin E deficiencies can cause serious neurological abnormalities. Also tocopherols react with and destroy the most reactive forms of oxygen, protecting unsaturated fatty acids from oxidation⁵. It is absolutely necessary that patients with the short bowel syndrome receive the essential fat soluble vitamins to avoid serious health problems. The fat soluble vitamins, A, D, E, and K are entirely hydrophobic compounds and are soluble only in lipids⁵. The nonpolar structure of the vitamins permit them from not interacting with water. The fat soluble vitamins can be readily incorporated into the hydrophobic interior of liposomes in a manner similar to their packaging into chylomicrons by intestinal epithelial cells.

Rationale for the Incorporation of Cobalamin (vitamin B₁₂) into the Aqueous Compartment of Liposomes for its Delivery into the Blood of Patients With the Small Bowel Syndrome: Vitamin B₁₂ is not synthesized by humans. The major source of vitamin B₁₂ is from the diet⁴. Intrinsic factor, a glycoprotein produced by the gastric parietal cells, is required for B₁₂ absorbtion⁴. It complexes with B₁₂ (the extrinsic factor) and facilitates the absorption of the vitamin by cells of the of the ileum⁴.

It was mentioned earlier that patients who have had their ileum resected cannot absorb vitamin B₁₂. The malabsorbtion of vitamin B₁₂ causes hemopoietic problems in patients with the short bowel syndrome. The hemopoetic problems caused by a B₁₂ deficiency are identical to those observed in a folate deficiency secondary to (i.e., caused by) the B₁₂ deficiency⁴. The N⁵-methytetrahydrofolate cannot be converted to free FH₄. Essentially, all of the folate becomes “trapped” as the N⁵-methyl derivatives. As the FH₄ pool is exhausted, deficiencies of the tetrahydrofolate derivatives needed for purine and dTMP biosynthesis develop leading to the characteristics of megaloblastic anemia⁴. B₁₂ deficiencies also cause neurological problems. It is absolutely necessary that patients who have the small bowel syndrome receive vitamin B₁₂ therapy periodically to prevent serious health problems. Vitamin B₁₂ is a water soluble vitamin and can be readily “trapped” in the aqueous compartment of liposomes and delivered directly to the liver where it is stored.

Rationale for the Incorporation of Metal Ions (Ca²⁺, Mg²⁺, and Zn²⁺) into the Aqueous Compartment of Liposomes for their Delivery into the Blood of Patients with the Small Bowel Syndrome. According to the literature, patients with the short bowel syndrome do not adequately absorb the metal ions, Ca²⁺, Mg²⁺, and Zn^{2+ 1-3}. All of these ions are essential to the well being of all humans and must be part of their dietary intake. The body does not synthesize ions. They represent only a miniscule fraction of the weight of the human body. However, they are essential to human life because they are essential to the function of specific enzymes. These ions function as metal cofactors in the catalysis of many different biochemical actions that occur in the body. Calcium (Ca²⁺) is also involved in hormone action and blood clotting. Calcium deficiency in the adult can result in bone loss. Magnesium activates many enzymes and also form a complex with ATP. Magnesium deficiencies can cause nausea, muscle weakness, irritability, and mental derangement. Zinc deficiency can cause dwarfism, loss of appetite, growth retardation, and hypogonadism.

Patients who have had their ileum resected are at great risk for malnutrition. They require ongoing non-parenteral nutritional support to replace the nutrients they cannot absorb. In addition, any single nutritional deficit predisposes them to a host of serious medical conditions, for example, infections, reproductive failure, anemia, neurological abnormalities, growth retardation, skin disorders, and etc. Therefore, it is absolutely necessary that patients with the short bowel syndrome receive daily or periodically all of the nutrients they cannot absorb from their diets. It is paramount that pediatric patients with the small bowel syndrome receive the nutrients they cannot absorb to avoid retardation in growth and reproduction. The current art of liposome technology can provide a safe, effective, and efficient vehicular means of delivering the nutrients that cannot be absorbed in patients with the short bowel syndrome. The nutrients can be commercially packaged into liposomes singly, in various combinations, or all of them together and delivered simultaneously into the blood. The state of art of liposome technology has allowed it to develop oral foams¹⁴⁻¹⁵. There are areas in the mouth or the back of the throat that will absorb the nutrient-loaded liposome foam directly into the blood.

The invention focuses on the delivery of certain nutrients via liposomes to patients with the short bowel syndrome. However, liposomes can be used to deliver the same or other nutrients to patients who have a diseased or a compromised gut.

### REFERENCES

1. Way, Lawrence W., M.D.: Current Surgical Diagnoses and Treatment. 10th. Edition. Norwalk, Conn., Appleton and Lang, 1994, pp. 620-622, 157-168.

2. Schwartz, Seymour I., M.D., Shires, Tom G., M.D., Spencer, Frank C., M.D.: Principles of Surgery. 5th Edition. New York, McGraw-Hill, Inc., 1989, pp. 1194, 1216-1218.

3. Wyngaarden, James B., M.D. and Smith, Lloyd H., Jr., M.D.: Cecil Textbook of Medicine. 18th Edition. Volume 1. Philadelphia, W. B. Saunders Company, 1988, pp. 732-734, 742-743, 745, 748-749, 751.

4. Marks, Dawn B., Marks, Allan D., M.D. Smith, Colleen M. Ph.D.: Basic Medical Biochemistry. New York, Lippincott Williams and Wilkins, 1996, pp. 11, 62, 130-131, 488, 494-495, 525, 521, 528-529, 531, 538-539, 541-542, 545, 547, 618-620.

5. Lehninger, Albert L., Nelson, David L., and Cox, Michael M. Principles of Biochemistry. 2nd. Edition. Worth Publishers, Inc. New York, 1993, PP 240-242, 254-256, 258-260, 481-482, 655, 674-675.

6. Stryer, Lubert.: Biochemistry. 3rd. Edition. New York, W. H. Freeman and Company, 1988. pp. 290-291.

7. Science and Technology. 8th. Edition. Volume 10. New York, McGraw Hill, 1997, pp.119-120.

8. Johnson, George B., Ober, William C.: Biology. 2nd. Edition. St. Louis, Mo., Times/Mirror/Mosby College Publishing, 1989, pp. 109-110.

9. Kim. S., et al., “Preparation of Multivesicular Liposomes”.Biochem. Biophys. Acta. 728 339-348 (1983).

10. Lasic, D. D., “liposomes: Synthetic Lipid Microsperes Servre as Multipurpose Vehicles for the Delivery of Drugs, Genetic Material and Cosmetics, ” Am. Sci. 80, 20-31 (1992).

11. Katre, N. V. et al., “A Multivesicular Lipid-Based, Sustained-Rlease System for the Delivery of Therapeutic Proteins, ” Pharmacol. Eng. pp. 8-18 (March-April 1999).

12. Ye, Q., Katre, N. and Sankaram, “Modulation of Drug Loading in Multivesicular Liposomes,” U.S. Pat. No. 6,106,858 (2000).

13. Pepper, C., Patel, M., and Hartounian, “CGMP Manufacturing Scale-Up of a Multivesicular Lipid-Based Drug Delivery System, ” Pharmacol. Eng. pp. 8-18 (March-April 1999).

14. Ye, Q. et al., “Depofoam Technology: A Vehicle for Controlled Delivery of Protein and Peptide Drugs”, J. Controlled Release 64, 155-166 (2000).

15. Spector, M. S., Zasadzinski, J. A., and Sankaram, M. B., “Topology of Multivesicular Liposomes: A model Biliquid Foam”, Langmuir 12, 4704-4708 (1996).

What is claimed is new and desired to be protected by the Letters Patent is set forth in the appended claims:

1. A method utilizing liposomes as non-parenteral vehicles for the delivery of nutrients into the blood of patients who have diseased or traumatized small bowels, and those who have undergone massive resections of the small bowel with the removal of the ileum.

2. The use of liposomes as non-parenteral vehicles for the delivery of the nutrients, cholesterol, linoleate, 18:2 (Cis Δ^{9,12}), α linolenate, 18:3 (Cis Δ^{9,12,15}) cobalamin (vitamin B₁₂), fat soluble vitamins (A, D, E, and K), and the metal ions (Ca²⁺,Mg²⁺, and Zn²⁺) into the blood of patients with the small bowel syndrome.

3. A method utilizing liposomes as non-parenteral vehicles for the delivery of the nutrients, cholesterol, linoleate 18:2 (Cis Δ^{9,12}), α linolenate 18:3 (Cis Δ^{9,12,15}), cobalamin, (vitamin B₁₂), fat soluble vitamins (A, D, E, and K), and the metal ions (Ca²⁺, Mg²⁺, and Zn²⁺) into the blood of patients who have had their distal small bowel (ileum) resected.
